(19) United States
(12) Patent Application Publication
(10) Pub. No.: US 2009/0105749 A1
(43) Pub. Date: Apr. 23, 2009

(54) Insertion and extraction tools for lacrimal implants

(75) Inventors: Eugene de Juan, San Francisco, CA (US); Cary J. Reich, Los Gatos, CA (US); Stephen Boyd, Murrieta, CA (US); Alan R. Rapacki, Redwood City, CA (US); Robert W. Shimizu, Laguna Niguel, CA (US); Valery Rubinchik, Richmond (CA); Charles Richard Kjellbotn, Qualicum Beach (CA)

Correspondence Address: SCHWEGMAN, LUNDBERG & WOESSNER, P.A., P.O. BOX 2938, MINNEAPOLIS, MN, 55402, US

(73) Assignee: QLT Plug Delivery, Inc. - QPDI, Menlo Park, CA (US)

(21) Appl. No.: 12/231,984

(22) Filed: Sep. 5, 2008

Related U.S. Application Data
(60) Provisional application No. 60/970,840, filed on Sep. 7, 2007.
(60) Provisional application No. 60/970,696, filed on Sep. 7, 2007.
(60) Provisional application No. 60/970,720, filed on Sep. 7, 2007.
(60) Provisional application No. 60/970,699, filed on Sep. 7, 2007.
(60) Provisional application No. 60/970,807, filed on Sep. 7, 2007.
(60) Provisional application No. 60/970,820, filed on Sep. 7, 2007.

(51) Int. Cl. A61F 9/007; A61B 17/28

(52) U.S. Cl. 606/206; 604/59; 606/205; 604/500

(57) ABSTRACT

Insertion and extraction tool, systems, and methods for use with lacrimal implants. An insertion tool is disclosed that includes a proximal end, a distal end, and a tool body therebetween. The distal end includes a mechanical coupling to receive a cartridge preloaded with a lacrimal implant, and a plunger configured to dispense the lacrimal implant from a preloaded cartridge.

### CLAIM OF PRIORITY

Portions of this non-provisional application claim the benefit of priority under 35 U.S.C. § 119(e) to U.S. Provisional Patent Application Ser. No. 60/970,840 filed on Sep. 7, 2007, the specification of which is herein incorporated by reference in its entirety.

### CROSS-REFERENCES TO RELATED APPLICATIONS

The present application is related to the following: U.S. patent application Ser. No. 11/695,537 filed on Apr. 2, 2007, titled “Drug Delivery Methods, Structures, And Compositions For Nasolacrimal System”; U.S. patent application Ser. No. 11/695,545 filed on Apr. 2, 2007, titled “Nasolacrimal Drainage System Implants For Drug Therapy”; U.S. Patent Application Ser. No. 60/970,696 filed on Sep. 7, 2007, titled “Expandable Nasolacrimal Drainage System Implants”; U.S. Patent Application Ser. No. 60/970,720 filed on Sep. 7, 2007, titled “Manufacture Of Swellable Nasolacrimal Drainage System Implants”; U.S. Patent Application Ser. No. 60/970,699 filed on Sep. 7, 2007, titled “Manufacture Of Drug Cores For Sustained Release Of Therapeutic Agents”; U.S. Patent Application Ser. No. 60/970,807 filed on Sep. 7, 2007, titled “System And Methods For Detection Of Nasolacrimal Devices”; and U.S. Patent Application Ser. No. 60/970,820 filed on Sep. 7, 2007, titled “Multiple Drug Delivery Systems and Combination Drugs With Punctal Implants”; all of which are incorporated herein by reference in their entirety.

### BACKGROUND OF THE INVENTION

The present application is related to lacrimal implants for use in or near the nasolacrimal drainage system, and more specifically to insertion and extraction tools for use with lacrimal implants, such as punctal implants including punctal or punctum plugs.

A variety of challenges face patients and physicians in the area of ocular drug delivery. In particular, the repetitive nature of the therapies (multiple injections, instilling multiple eye drop regimens per day), the associated costs, and the lack of patient compliance may significantly impact the efficacy of the therapies available, leading to reduction in vision and many times blindness.

Patient compliance in taking the medications, for example instilling the eye drops, can be erratic, and in some cases, patients may not follow the directed treatment regime. Lack of compliance can include, failure to instill the drops, ineffective technique (instilling less than required), excessive use of the drops (leading to systemic side effects), and use of non-prescribed drops or failure to follow the treatment regime requiring multiple types of drops. Many of the medications may require the patient to instill them up to 4 times a day.

In addition to compliance, the cost of at least some eye drop medications is increasing, leading some patients on limited incomes to be faced with the choice of buying basic necessities or instead getting their prescriptions filled. Many times insurance does not cover the total cost of the prescribed eye drop medication, or in some cases eye drops containing multiple different medications.

Further, in many cases, topically applied medications have a peak ocular effect within about two hours, after which additional applications of the medications should be performed to maintain the therapeutic benefit. In addition, inconsistency in self-administered or ingested medication regimes can result in a suboptimal therapy. PCT Publication WO 06/014434 (Lazar), which is incorporated herein by reference in its entirety, may be relevant to these and/or other issues associated with eye drops.

One promising approach to ocular drug delivery is to place an implant that releases a drug in tissue near the eye. Although this approach can offer some improvement over eye drops, some potential problems of this approach may include implantation of the implant at the desired tissue location, retention of the implant at the desired tissue location, and sustaining release of the drug at the desired therapeutic level for an extended period of time.

One problem with lacrimal implants, such as a punctal or punctum plug, is the difficulty inserting them into the punctum. The implants are very small and may not be inserted into punctum fully, such that they fall out easily. The implants may also be difficult to remove from the punctum.

In light of the above, it would be desirable to provide an improved insertion and/or extraction tool for lacrimal implants that overcome at least some of the above mentioned shortcomings.

### EXEMPLARY ASPECTS AND FEATURES OF THE INVENTION

The present invention provides improved insertion and extraction tools for use with an implant in a punctum of a patient.
1. An insertion tool for insertion of an implant into a punctum of a patient or subject includes a tool body having a distal portion configured to hold the implant on an outer implant surface, the distal portion having an inner lumen with an internal depth stop, and a plunger slidable within the inner lumen to engage and dispense the implant, the plunger having a stop configured to engage with the internal depth stop, wherein the engagement of the stop and the internal depth stop limits an insertion depth of the implant into the punctum.
2. The insertion tool according to aspect 1, wherein a distal end of the tool body optionally includes a tissue stop configured to engage tissue proximate the punctum.
3. The insertion tool according to aspects 1 and 2, wherein the tissue stop is optionally is made of one or both of a clear material and a magnifying material.
4. The insertion tool according to aspects 1-3, wherein the tissue stop optionally includes a magnifying geometry.
5. The insertion tool according to aspects 1-4, optionally including a tip couplable to the body proximate the implant, the tip having an inner lumen sized for the implant to slide therethrough.
6. The insertion tool according to aspects 1-5, wherein the tip optionally includes one or more slots configured to slideably fit one or more protrusions of the implant.
7. The insertion tool according to aspects 1-6, wherein the tip optionally is sized to fit at least partially within, and dilate, the punctum.
8. The insertion tool according to aspects 1-7, wherein the tip optionally is angled or curved relative to a longitudinal body axis, the angle or curve facilitating placement of the implant in a superior punctum.
9. The insertion tool according to aspects 1-8, optionally including a retractable sheath configured to surround a portion of the implant.
10. A lacrimal implant insertion tool for use with a lacrimal implant includes a tool body having a proximal handle, a distal end, and an axis therebetween. The tool body includes an implant receptacle releasably supporting, on at least one outer implant surface, the lacrimal implant relative to the handle, such that the lacrimal implant is advanceable distally into a canalicular lumen by manipulation of the handle, and a tissue-engagement stop surface, the stop surface being distally oriented and configured to engage an anteriorly oriented tissue surface to inhibit distal insertion of the lacrimal implant beyond a target insertion depth.
11. The lacrimal implant insertion tool according to aspect 10, wherein implant receptacle optionally includes a sheath.
12. The lacrimal implant insertion tool according to aspects 10 and 11, wherein the distal end optionally includes a punctum dilator having a conical portion.
13. A lacrimal implant insertion system for treatment of one or more tissues near a punctum of a patient, includes a self-dilating lacrimal implant, and an insertion tool having a proximal handle, a distal implant receptacle, and an axis therebetween, the implant receptacle releasably supporting the lacrimal implant such that the lacrimal implant is advanceable distally into the canalicular lumen by manipulation of the handle, the insertion tool including a tissue-engagement stop surface, the stop surface being distally oriented and configured to engage the anteriorly oriented tissue surface so as to inhibit distal insertion of the lacrimal implant beyond a target insertion depth.
14. The lacrimal implant insertion system according to aspect 13, wherein the implant receptacle optionally includes a sheath.
15. The lacrimal implant insertion system according to aspects 13 and 14, wherein the sheath optionally includes an inclined surface configured to dilate the punctum.
16. A method of inserting an implant into a punctum of a patient or subject using an insertion tool includes advancing the implant distally into the punctum, engaging a tissue stop of the insertion tool against a tissue surface of the punctum so as to inhibit distal movement of the insertion tool, and detaching the implant from the insertion tool while the tissue stop engages the tissue surface and while the implant is aligned axially relative to the tissue stop so that the implant is implanted at a target depth within the canalicular lumen.
17. The method according to aspect 16, wherein the detaching the implant from the insertion tool optionally includes depressing a plunger to engage a wire to release the implant from the insertion tool.
18. The method according to aspects 16 and 17, optionally including supporting the lacrimal implant on at least one outer implant surface with the insertion tool.
19. A method of inserting an implant into a punctum of a patient using an insertion tool includes placing a tissue stop of the insertion tool proximate the punctum, moving a plunger within the insertion tool forward, thereby inserting the implant into the punctum, and stopping the plunger movement when a stop on the plunger engages an internal depth stop of the insertion tool, wherein the engagement of the stop and internal depth stop limits the depth of insertion of the implant into the punctum.
20. The method according to aspect 19, optionally includes supporting the lacrimal implant on at least one outer implant surface with a sheath.
21. The method according to aspects 19 and 20, optionally including dilating the punctum with the sheath.
22. An extraction tool for extraction of an implant from a punctum of a patient or subject includes a distal portion, wherein the distal portion includes an extraction feature to engage a complimentary extraction feature of the implant.
23. The extraction tool according to aspect 22, wherein the distal portion optionally includes one or more angled tips configured to engage one or more protrusions extending from the implant.
24. The extraction tool according to aspects 22 and 23, optionally including one or more tips extending radially from the distal end to engage one or more grooves of the implant.
25. The extraction tool according to aspects 22-24, wherein the distal portion optionally includes a hook feature configured to engage one of a loop or handle of the implant.
26. An extraction tool for extraction of an implant from a punctum of a patient or subject includes an extraction tool body having a distal portion, and a suction device configured to provide a suction force to the extraction tool body, wherein the distal portion of the extraction tool includes an inner lumen extending to a tip of the distal portion, and wherein the tip is configured to engage the punctum and apply the suction force to extract the implant.
27. The extraction tool according to aspect 26, wherein the tip of the distal portion optionally is configured for insertion within the punctum to apply the suction force within the punctum.
28. The extraction tool according to aspects 26 and 27, wherein the tip of the distal portion optionally is configured for insertion within the punctum, and the tip of the distal portion includes a diameter less than or equal to a diameter of the implant to apply the suction force to the implant.
29. An implant insertion tool for use with a lacrimal implant includes a proximal end, a distal end, and a tool body therebetween. The distal end includes a mechanical coupling to receive a cartridge preloaded with a lacrimal implant and a plunger configured to dispense the lacrimal implant from a preloaded cartridge.
30. The insertion tool according to aspect 29, wherein the cartridge optionally engages an outer surface of the lacrimal implant and contains an inner lumen. The plunger has a diameter greater than or equal to a diameter of a plunger receiving surface of the lacrimal implant and the plunger slides within the inner lumen and engage and dispense the lacrimal implant from the cartridge.
31. The insertion tool of according to aspects 29 and 30, wherein the proximal end of the insertion tool optionally includes an insertion facilitating portion.
32. The insertion too according to aspects 29-31, wherein the insertion facilitating portion optionally includes a curvature substantially similar to a curvature of at least a portion of the lacrimal implant.
33. The insertion tool according to aspects 29-32, optionally including a living hinge coupled to the plunger. The living hinge causes the plunger to dispense the lacrimal implant.
34. A system for treatment of an eye includes a lacrimal implant, a cartridge configured to hold the lacrimal implant, and a lacrimal implant insertion tool for use with the lacrimal implant. The insertion tool includes a proximal end, a distal end, and a tool body therebetween. The distal end includes a mechanical coupling to receive a cartridge preloaded with a lacrimal implant and a plunger configured to dispense the lacrimal implant from a preloaded cartridge.
35. The system according to aspect 34, wherein the plunger of the insertion tool optionally has a diameter greater than or equal to a diameter of a plunger receiving surface of the lacrimal implant.
36. The system according to aspects 34 and 35, wherein the lacrimal implant optionally includes a drug eluting portion and a plug portion surrounding at least a portion of the drug eluting portion. The plunger diameter is greater than or equal to a diameter of the plug portion, and the plunger engages the plug portion to dispense the lacrimal implant.
37. The system according to aspects 34-36, wherein the cartridge is optionally rotatable relative to the distal end of the insertion tool.
38. The system according to aspects 34-37, wherein the proximal end of the insertion tool optionally includes an insertion facilitating portion, The insertion facilitating portion includes a curvature substantially similar to a curvature of at least one of the drug eluting portion and the plug portion of the lacrimal implant.
39. An implant insertion tool for use with a lacrimal implant includes a proximal end, a distal end, and a tool body therebetween. The distal end includes a forceps that are sized to engage the lacrimal implant on an outer surface of the implant. The insertion tool is configured to lock a position of the forceps when the lacrimal implant is so engaged.
40. The insertion tool according to aspect 39, optionally including a collar to slidably engage the forceps to cause the forceps to open and close.
41. The insertion tool according to aspects 39 and 40, optionally including a lever located on the tool body. Wherein manipulating the lever causes the collar to slidably engage the forceps.
42. The insertion tool according to aspects 39-41, wherein an end of each arm of the forceps optionally includes a groove substantially perpendicular to the forceps arm. The grooves are sized to receive at least a portion of the lacrimal implant when the forceps are closed.
43. The insertion tool according to aspects 39-42, wherein at least one of the forceps arms of the insertion tool optionally includes a stop to engage an end of the lacrimal implant and inhibit movement of the lacrimal implant relative to the forceps.
44. The insertion tool according to aspects 39-43, wherein the proximal end of the insertion tool optionally includes an insertion facilitating portion.
45. The insertion tool according to aspects 39-44, wherein the proximal end of the insertion tool optionally includes a second forceps configured to extract the lacrimal implant from the punctum.
46. The insertion tool according to aspects 39-45, wherein the forceps of embodiments 39-45 are optionally detachable from the tool body.
47. A method of inserting an implant using an insertion tool includes preloading a lacrimal implant into a cartridge, and dispensing the lacrimal implant from the cartridge to insert the lacrimal implant into a punctum.
48. The method according to aspect 47, optionally including engaging an outer surface of the lacrimal implant to releasably support the lacrimal implant.
49. The method according to aspects 47 and 48, wherein the dispensing the lacrimal implant optionally includes dispensing the lacrimal implant from the cartridge using a plunger.
50. The method according to aspects 47-49, optionally includes manipulating a living hinge on the insertion tool to engage the lacrimal implant with the plunger.
51. A method of inserting an implant using an insertion tool includes engaging an outer surface of the lacrimal implant with a forceps, locking a forceps position when the outer surface of the lacrimal implant is engaged, and advancing the lacrimal implant into a punctum.
52. The method according to aspect 51, optionally including slidably engaging arms of the forceps with a collar to open and close the forceps.
53. The method according to aspects 51-52, wherein the slidably engaging arms of the forceps with a collar optionally includes manipulating a lever to cause the collar to slidably engage the arms of the forceps.
54. The method according to aspects 51-53, optionally including receiving the lacrimal implant into a groove on a forcep arm when the forceps are closed. The groove is substantially perpendicular to the forcep arm and is sized to receive the lacrimal implant.
55. The method according to aspects 51-54, wherein the advancing the lacrimal implant into the punctum optionally includes engaging an end of the lacrimal implant with a stop on a forcep arm to inhibit movement of the lacrimal implant relative to the forcep arm.
56. The method according to aspects 51-55, optionally including changing the forceps of the insertion tool to fit a geometry of the lacrimal implant.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIGS. 1A and 1B show anatomical tissue structures of the eye suitable for use with various implants, according to embodiments of the present invention.

FIG. 2 shows an insertion tool to insert an implant into the punctum with a plunger that can be depressed, according to an embodiment of the present invention.

FIG. 3 shows an insertion tool to insert an implant into the punctum with a plunger that can slide, according to an embodiment of the present invention.

FIG. 4 shows an insertion tool to insert an implant into the punctum with a sheath that retracts proximally, according to an embodiment of the present invention.

FIGS. 5A and 5B show an insertion tool 500 to insert an implant into the punctum having a tissue stop and internal depth stop, according to an embodiment of the present invention.

FIG. 6 shows an insertion tool to insert an implant having protrusions into the punctum, according to an embodiment of the present invention.

FIG. 7A shows an implant wing folding device 700, according to an embodiment of the present invention.

FIGS. 7B-D show the implant wing folding device 700 in use.

FIGS. 8A-8C show different lead-in designs and dilators that may be used with many of the insertion tool embodiments, according to embodiments of the present invention.

FIG. 9A shows a distal end of an insertion tool that includes a lead-in, according to an embodiment of the present invention.

FIG. 9B shows a distal end of an insertion tool that includes a curved lead-in, according to an embodiment of the present invention.

FIGS. 10A and 10B show loading an implant in an insertion tool, according to an embodiment of the present invention.

FIG. 11A is a top view showing an implant that includes one or more protrusions or wings that may be grasped by an extraction tool, according to an embodiment of the present invention.

FIG. 11B is a side view of FIG. 11A showing the implant and extraction tool.

FIG. 12A is a top view showing an implant that includes one or more grooves into which an extraction tool is inserted for removal of the implant, according to an embodiment of the present invention.

FIG. 12B is a side view of FIG. 12A showing the implant and extraction tool, according to an embodiment of the present invention.

FIG. 13 shows an implant having a loop or handle on a top portion that can be grasped by an extraction tool for removal, according to an embodiment of the present invention.

FIGS. 14A-14C show suction extraction tools, according to embodiments of the present invention.

FIG. 15 shows an extraction tool that includes a helical filament for implant removal, according to embodiments of the present invention.

FIG. 16 shows an extraction tool that is a “flusher” device, according to embodiments of the present invention.

FIG. 17 shows one embodiment of an extraction tool that is a “pusher” device, according to embodiments of the present invention.

FIG. 18 shows another embodiment of an insertion tool for use with an implant.

FIG. 19 shows a view of an embodiment of the distal end of the insertion tool in FIG. 18.

FIG. 20 shows a view of an embodiment of the proximal end of the insertion tool in FIG. 18.

FIG. 21 shows another embodiment of an insertion tool for use with an implant.

FIG. 22 shows a view of an embodiment of the distal end of the insertion tool.

### DETAILED DESCRIPTION

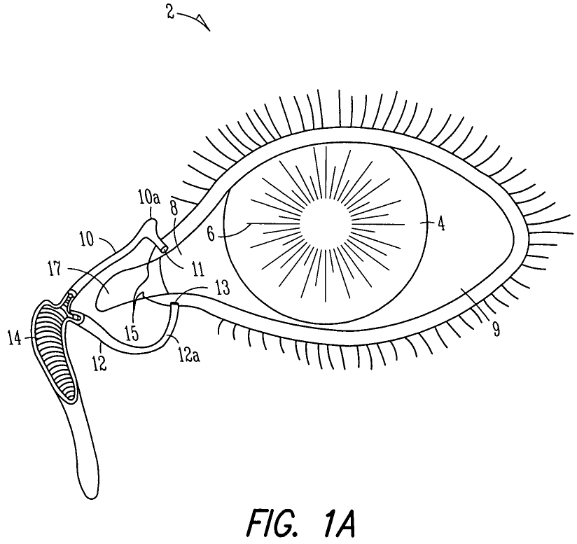

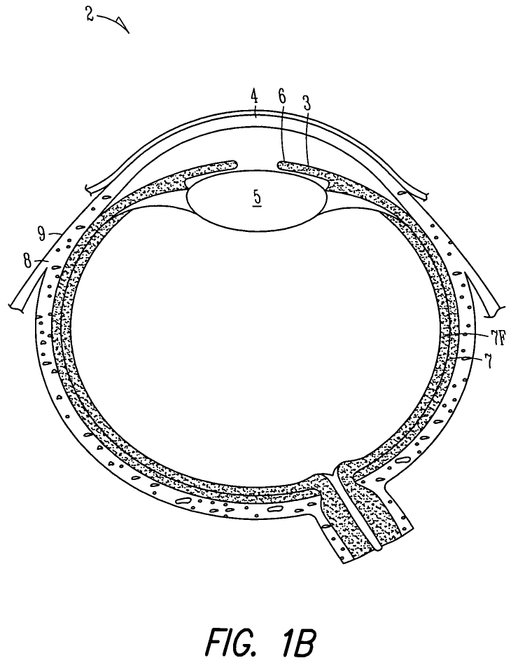

FIGS. 1A and 1B show anatomical tissue structures of an eye 2 suitable for treatment with implants, according to an embodiment of the present invention. Eye 2 includes a cornea 4 and an iris 6. A sclera 8 surrounds cornea 4 and iris 6 and appears white. A conjunctival layer 9 is substantially transparent and disposed over sclera 8. A crystalline lens 5 is located within the eye. A retina 7 is located near the back of eye 2 and is generally sensitive to light. Retina 7 includes a fovea 7F that provides high visual acuity and color vision. Cornea 4 and lens 5 refract light to form an image on fovea 7F and retina 7. The optical power of cornea 4 and lens 5 contribute to the formation of images on fovea 7F and retina 7. The relative locations of cornea 4, lens 5 and fovea 7F are also important to image quality. For example, if the axial length of eye 2 from cornea 4 to retina 7F is large, eye 2 can be myopic. Also, during accommodation, lens 5 moves toward cornea 4 to provide good near vision of objects proximal to the eye.

The anatomical tissue structures shown in FIG. 1A also include the lacrimal system, which includes an upper canaliculus 10 and a lower canaliculus 12, collectively the canaliculae, and the naso-lacrimal duct or sac 14. The upper and lower canaliculae terminate in an upper punctum 11 and a lower punctum 13, also referred to as punctal apertures. The punctal apertures are situated on a slight elevation at the medial end of the lid margin at the junction 15 of the ciliary and lacrimal portions near the medial canthus 17. The punctal apertures are round or slightly ovoid openings surrounded by a connective ring of tissue. Each of the punctal openings 11, 13 leads into a vertical portion 10a, 12a of the respective canaliculus before turning horizontally to join its other canaliculus at the entrance of a lacrimal sac 14. The canaliculae are tubular and lined by stratified squamous epithelium surrounded by elastic tissue which permits the canaliculus to be dilated.

### Insertion

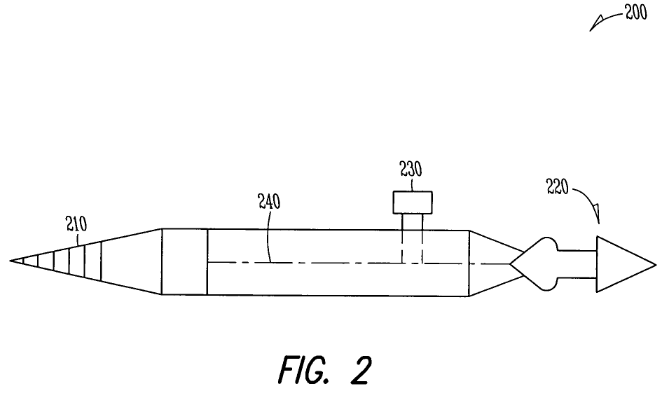

FIGS. 2, 3 and 4 show embodiments of insertion tools that can be used to insert different lacrimal implants, which include punctal implants such as punctal or punctum plugs. In other embodiments, the implant is a drug delivery implant that includes a drug insert and a commercially available lacrimal implant that can accommodate the drug insert. The drug insert can be adapted to be placed in the bore of the lacrimal implant, and can be held in place via an interference fit between the outer diameter of the drug insert and the inner diameter of the silicone plug bore. The assembled system can be packaged and sterilized and delivered to the physician in this configuration. Many embodiments of lacrimal implants suitable with the present application are disclosed in U.S. patent application Ser. No. 11/695,545, filed on Apr. 2, 2007, titled “Nasolacrimal Drainage System Implants for Drug Therapy”, which is incorporated herein by reference in its entirety. In some embodiments, the lacrimal implant may be a commercially available punctum plug.

FIG. 2 shows an insertion tool 200 to insert an implant into the punctum with a plunger 230 that can be depressed, according to an embodiment of the present invention, Insertion tool 200 includes a dilator 210 that can be inserted into the punctum to pre-dilate the punctum prior to insertion of an implant. An implant 220 can be pre-loaded onto tool 200 prior to dilation of the punctum. An internal wire 240 can be connected to implant 220 to retain or releasably support the implant 220. Following pre-dilation of the punctum with dilator 210, tool 200 can be used to insert the implant 220 into the punctum by distally advancing the implant 220 into a canalicular lumen through manipulation of the handle. In some examples, the implant 220 is shaped to be self-dilating. Descriptions of self-dilating lacrmial implants can be found in Rapacki et al., co-pending, commonly assigned, U.S. Patent Application Ser. No. 61/066,233 “Lacrimal Implants and Related Methods,” which is incorporated herein in its entirety. While implant 220 is positioned in the punctum, plunger 230 can be depressed to engage wire 240 and release implant 220 from tool 200. In some embodiments, wire 240 may comprise a sharpened needle tip that penetrates implant 220. Implant 220 may be any lacrimal implant made of a resilient material, for example silicone. In some embodiments, the lacrimal implant may also include a drug core, such that the drug core material contracts when the needle is removed.

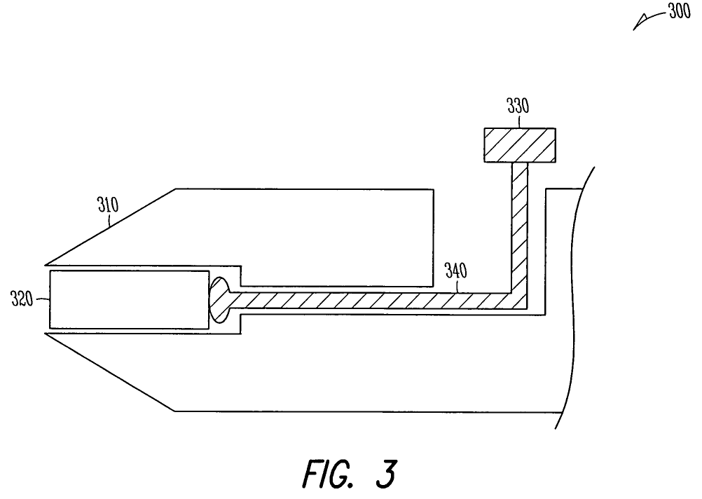

FIG. 3 shows an insertion tool 300 to insert an implant 320 into the punctum with a plunger that can slide, according to an embodiment of the present invention. Insertion tool 300 includes a dilator 310 with a conical section to dilate the punctum and a plunger 330 that can slide distally to advance implant 320 into the lumen. A shaft 340 is connected to plunger 330 to advance implant 320 distally when plunger 330 is advanced distally. While the punctum is dilated with dilator 310, plunger 330 can be advanced distally to place implant 320 in the canalicular lumen near the punctum. In many embodiments, a button can be depressed to advance distally the implant into the lumen, for example a button connected to shaft 340 with an intermediate mechanism.

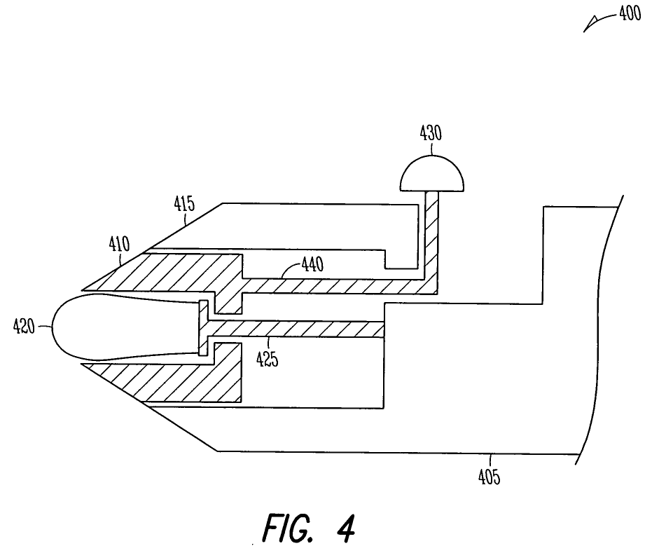

FIG. 4 shows an insertion tool 400 to insert an implant into the punctum with a sheath 410 that retracts to position the implant in the canalicular lumen, according to an embodiment of the present invention. The sheath 410 releasably supports the implant 420 on at least one outer implant surface. At least a portion of the sheath 410 is shaped to dilate the punctum. Sheath 410 is shaped to hold an implant 420 in a small profile configuration. Insertion tool 400 includes an annular structure 415, which can comprise a portion of a body 405 of insertion tool 400. Sheath 410 and annular structure 415 are shaped to dilate the punctum and often comprise proximally inclined surfaces to dilate the punctum. Implant 420, sheath 410 and annular structure 415 can be at least partially inserted into the punctum to place the implant in the canalicular lumen. Annular structure 415 is disposed over sheath 410 so that sheath 410 can be retracted and slide under annular structure 415. A stop 425 can be connected to body 405 to retain implant 420 at the desired depth within the canalicular lumen while sheath 410 is retracted proximally to expose implant 420.

Once implant 420 has been positioned in the canalicular lumen at the desired depth in relation to the punctum, sheath 410 is retracted to expose implant 420 at the desired location in the canalicular lumen. A plunger 430 can be used to retract sheath 410. A shaft 440 mechanically couples sheath 410 to plunger 430. Thus, retraction of plunger 430 in the proximal direction can retract sheath 410 in the proximal direction to expose implant 420 at the desired location in the canalicular lumen. Implant 420 can be any of the implants as described herein. Often, implant 420 will comprise a resilient member that expands to a large profile configuration when sheath 410 is retracted.

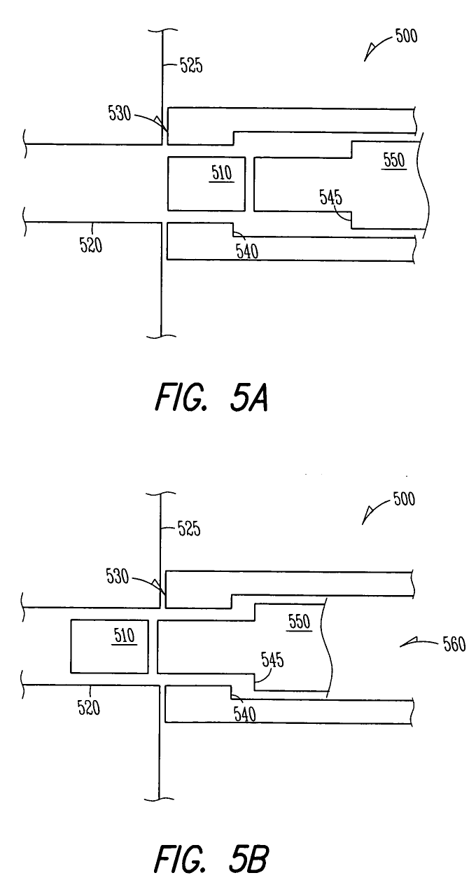

FIGS. 5A and 5B show another embodiment of an insertion tool 500 to insert an implant 510 into the punctum 520. The insertion tool 500 includes a tool body with an inner lumen having a tissue stop 530 at a distal end and an internal depth stop 540. The tissue stop 530 creates a datum on the tissue surface 525 from which the implant 510 can be inserted into the punctum 520. The internal depth stop 540 engages a stop 545 on a plunger 550 that limits the depth placement relative to the eyelid for the implant 510 within the punctum 520. The plunger is designed to engage and dispense the implant. The insertion tool 500 is designed to place the implant in the same location in the punctum so that the upper surface of the plug is positioned consistently with the eyelid. The insertion tool 500 is also designed to prevent excessive injection depth of the implant in the punctum. In use, the tissue stop 530 is placed proximate the punctum 520. The plunger 550 is moved forward 560 inserting the implant 510 into the punctum 520 until stop 545 engages internal depth stop 540. Then the insertion tool 500 is removed.

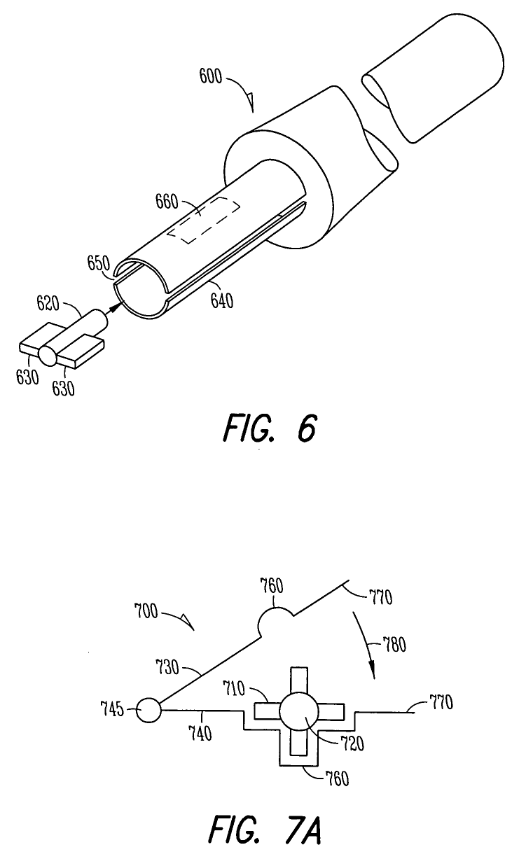

FIG. 6 show one embodiment of a distal end of an insertion tool 600 for use with an implant 620, such as a punctal plug, having one or more protrusions 630. The distal end of the insertion tool 600 has a delivery tube 640 that includes slots 650 on the sides to orient the implant 620 properly. To assist in this orientation, markings 660 may be placed on the outsides of the delivery tube indicating the proper orientation of the implant 620. For example, the markings may include directions for implantation, such as “toward eye” or “away from eye” or other helpful instructions. The protrusions 630 may be grasped with an extraction tool, such as forceps and or other instruments, to remove the implant 620 from the punctum. The insertion tool 600 may be made similar to intraocular lens (IOL) inserters, such as shown in U.S. Pat. No. 4,747,404, titled “Foldable Intraocular Lens Inserter”, which is incorporated herein by reference in its entirety.

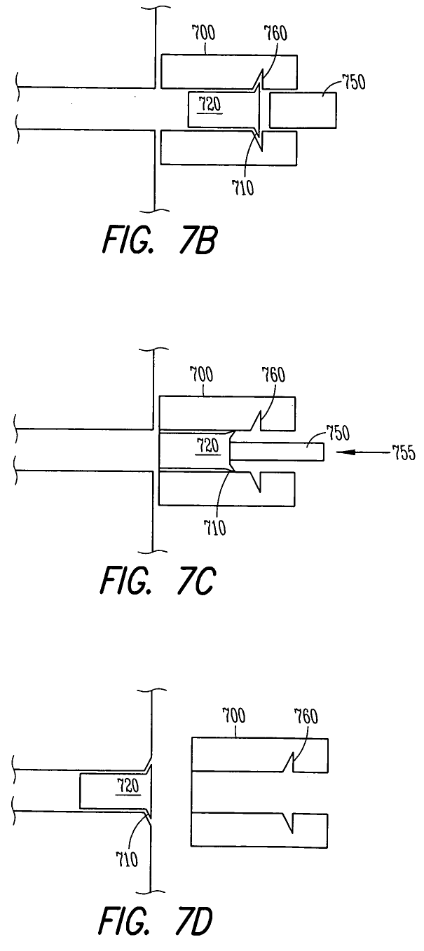

FIG. 7A shows one embodiment of an implant wing folding device 700. The wing folding device 700 may be used to fold or compress a depth registration head, such wings or protrusions 710 of an implant 720, so that the implant 720 may be loaded in a tube of an insertion tool, such as shown in FIGS. 7B-7D. The folding device 700 includes an upper portion 730 and a lower portion 740 coupled with a hinge 745. The upper and lower portions 730, 740 include various indentations 760 for the wings or protrusions 710 of the implant 720. The upper and lower portions 730, 740 and indentations 760 are designed to control the folding and/or compression force on the wings or protrusions 710 and the implant 720. Surfaces of the upper and lower portions 730, 740 and indentations 760 may include a lubricant 770 to aid in folding or compressing the wings or protrusions 710. The lubricant may also aid in inserting the folded or compressed implant 720 into the tube of the insertion tool. The implant should be made of a material that has a memory, so the once the implant leave the tube, it expands to its original shape. In use, the implant 720 is positioned between the upper and lower portions 730, 740 proximate the indentations 760. The upper and lower portions 730, 740 are then brought together 780, folding or compressing the wings or protrusions 710, and the implant 720 is then loaded into the insertion tool. The folding device 700 may be similar to intraocular lens (IOL) folding devices, such as shown in Brady et al., U.S. Pat. No. 5,947,974, titled “Folding Device And Method For An Intraocular Lens”, filed Dec. 9, 1997, which is incorporated herein by reference in its entirety.

FIGS. 7B-7D show that the inserter would cause the depth registration head, such wings or protrusions 710 to follow behind the implant 720 (the folder would allow these elements to trail the body of the plug in delivery). The wings or protrusions 710 are temporarily deformed (distorted) to allow to trail the body. In the free position the wings or protrusions 710 deploy to their natural (normal/static) position that allow for checking placement of the plug at the surface of the punctum. The silicone material of the plug has sufficient memory that it recovers after displacement within the tube. FIG. 7B shows the implant 720 in place with the folding device 700, with the wings or protrusions 710 positioned within the indentations 760. A piston or plunger 750 then pushes the implant forward 755, folding the wings or protrusions 710 back, such as shown in FIG. 7C. Once the wings or protrusions 710 clear the folding device 700 they can then expand to their open or flattened configuration, such as shown in FIG. 7D.

As discussed above, in many embodiments, the insertion tool may include a tip that is a dilator to dilate the punctum prior to insertion of the implant. The dilator may be positioned at either end of the insertion tool, for example, the insertion tool may be positioned on an end of the insertion tool that opposes the end loaded with the implant, such as shown in FIG. 2, or the dilator may be positioned on an end with the implant as part of the lead-in, such as shown in FIGS. 3 and 4.

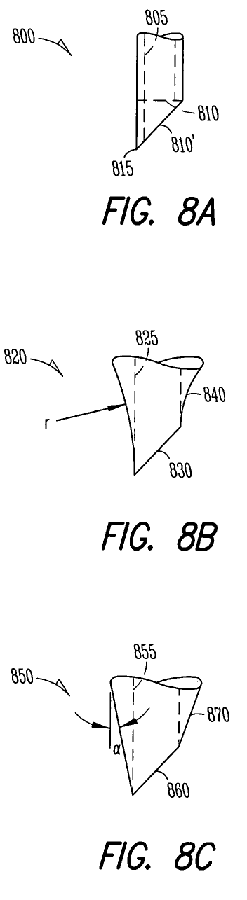

FIGS. 8A-8C show different embodiments of lead-in designs that may be used with many of the insertion tool embodiments described herein. FIG. 8A shows a tip or lead in 800 used as a hole guide that is inserted into the punctum prior to inserting the implant. The implant is delivered through an internal lumen 805 of the lead-in 800. The distal end of the lead-in 800 may be have a straight cut tip 810 or a beveled or angled cut tip 810′. Testing has shown beveled cut tip 810′ allowed easier entry of the lead-in 800 into the punctum over the straight cut tip 810. A slight radius 815 may be added to the point of the beveled cut tip 810′ so the device is less traumatic during insertion. In some embodiments, the lead-in may also be used as a dilator. FIG. 8B shows a lead-in 820 that includes a beveled cut tip 830 at a distal end and radiused sides 840; such that the radiused side 840 diameter r gradually increases along its length to dilate the punctum as well as create a guide for the insertion tube through an internal lumen 825. In another embodiment shown in FIG. 8C, lead in 850 includes a beveled portion 860 at a distal end and tapered sides 870 having an angle α to dilate the punctum as well as create a guide for the insertion tube through an internal lumen 855.

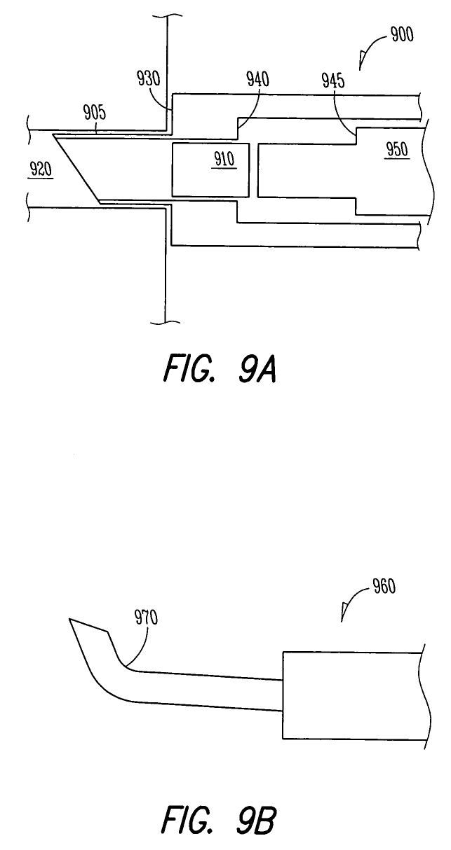

FIG. 9A shows one embodiment of a distal end of an insertion tool 900 that includes a lead-in 905 or tip that can be inserted into the punctum 920 prior to insertion of an implant 910. Insertion tool 900 also includes a tissue stop 930 at a proximal end and a tip or lead-in 905. In addition, insertion tool 900 may have an internal depth 940 that mates with stop 945 of plunger 950. As discussed above, lead-in 910 may have a dilator shape. In some embodiments, the lead-in is permanent on the insertion tool. In other embodiments, the lead-in may be removable, such that the size and shape of the lead-in selected depends on the punctum size. FIG. 9B shows one embodiment of a distal end of an insertion tool 960 that includes an angled or curved lead-in 970. The angled or curved lead-in may be desirable for easier placement of the implant in the superior punctum.

In one embodiment, a portion of the insertion tool proximate the tissue stop may be made of clear material, such as an acrylic material, so that the physician can visualize the tissue through the insertion tool and see the punctum. The clear material may also allow viewing of an implant while it is being implanted, and may also confirm that the implant is implanted properly. In another embodiment, the clear material may be a magnifying material and/or have a magnifying geometry, such as a spherical lens or angled lens, so that the punctum is more easily visualized.

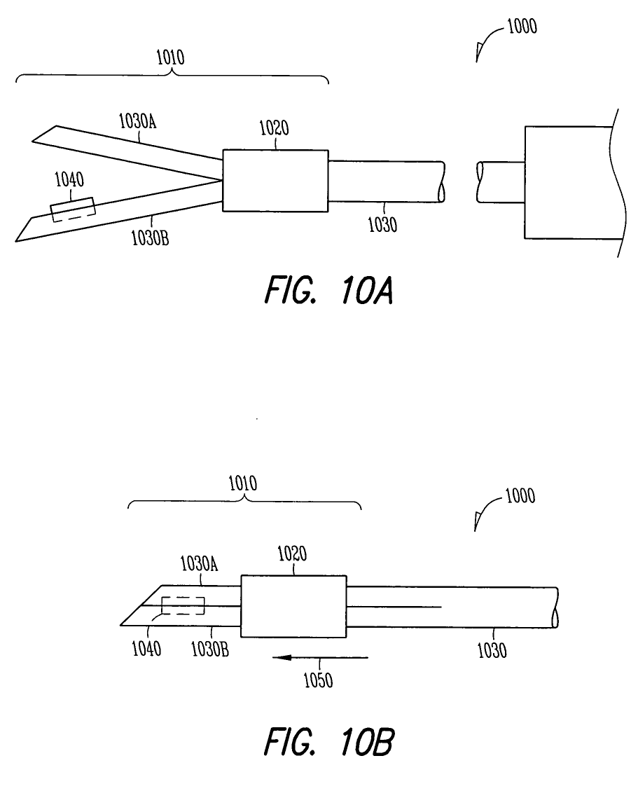

FIGS. 10A and 10B show one embodiment of loading an implant in an insertion tool 1000. The insertion tool 1000 includes a loading clamp 1010 distal portion having a sliding collar 1020 that is slid along a tube 1030 to load an implant 1040 in the insertion tool 1000. The tube 1030 has splitable portions 1030A and 1030B on a distal end. The implant 1040 is positioned within the splitable portions 1030A and 1030B and the sliding collar 1020 is advanced distally 1050 to close the splitable portions 1030A and 1030B together. The implant 1040 is then ready for implantation into a punctum. Once in place, the collar 1020 may act as a tissue stop. The collar 1020 may also be made of a clear material or a magnifying material, as discussed above.

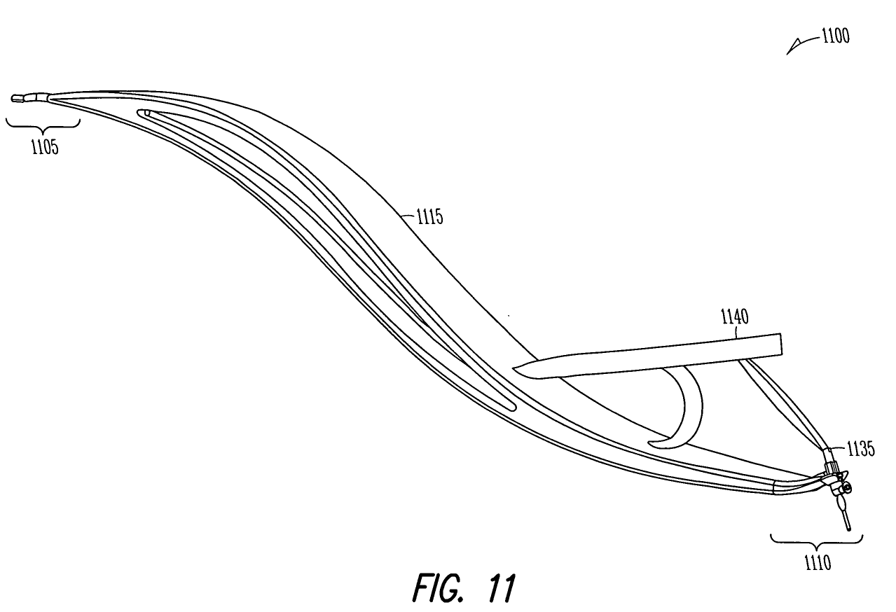

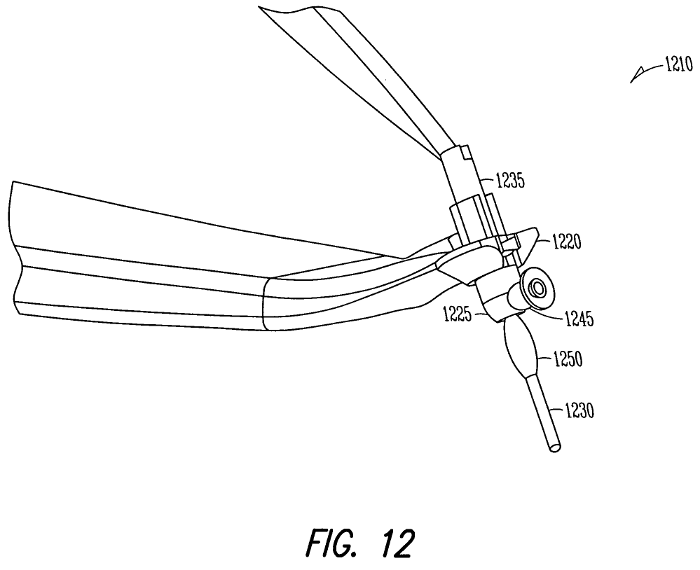

FIG. 11 shows another embodiment of an insertion tool 1100 for use with a lacrimal implant. The insertion tool 1100 includes a proximal end 1105, a distal end 1110, and a tool body 1115 therebetween. FIG. 12 shows a view of an embodiment of the distal end 1210. The distal end 1210 includes a mechanical coupling 1220 to receive a cartridge 1225. The cartridge 1225 is preloaded with a lacrimal implant 1230. In some embodiments, the cartridge 1225 is rotatable relative to the insertion tool. The cartridge 1225 releasably supports the lacrimal implant 1230. The lacrimal implant 1230 is quite small and may be pre-loaded into the cartridge 1225 while viewing under a microscope. The cartridge 1225 may be single use or reloadable with a new ocular implant after use.

In the embodiment shown, the lacrimal implant 1230 is an L-shaped self-dilating punctum plug. The punctum plug includes a drug eluting portion 1245 and a plug portion 1250 surrounding at least a portion of the drug eluting portion 1245. In the example in the Fig., the drug eluting portion 1245 is transverse to the plug portion 1250. A discussion of a self-dilating lacrimal implant may be found in the previously mentioned Rapacki et al. One of ordinary skill in the art would understand, upon reading this document, that a cartridge preloaded with other types of lacrimal implants are within the scope of the present invention. Different cartridges may be used for different types of lacrimal implants.

The cartridge 1225 engages an outer surface of the lacrimal implant 1930 and contains an inner lumen. The inner lumen has a curvature to match a curvature of at least a portion of the lacrimal implant 1230 to provide support to the lacrimal implant 1230. The distal end 1210 also includes a plunger 1235 that dispenses the lacrimal implant 1230 from the cartridge 1225. In some embodiments, the plunger 1235 has a diameter greater than, or equal to, a diameter of a plunger-receiving surface of the lacrimal implant 1230. In the example, the plunger-receiving surface is included in the plug portion 1250 of the implant. The plunger 1235 slides within the inner lumen and engages and dispenses the punctal implant 1230 from the cartridge 1225 and into the punctum.

Returning to FIG. 11, in some embodiments at least one of the proximal end 1105, the distal end 1110, and the tool body 1115 is formed by injection molding. In certain embodiments, the insertion tool 1100 includes a living hinge coupled to the tool body 1115 and the plunger 1135. Manipulating the living hinge (e.g., pressing the living hinge toward the tool body 1115) causes the plunger to dispense the punctal implant.

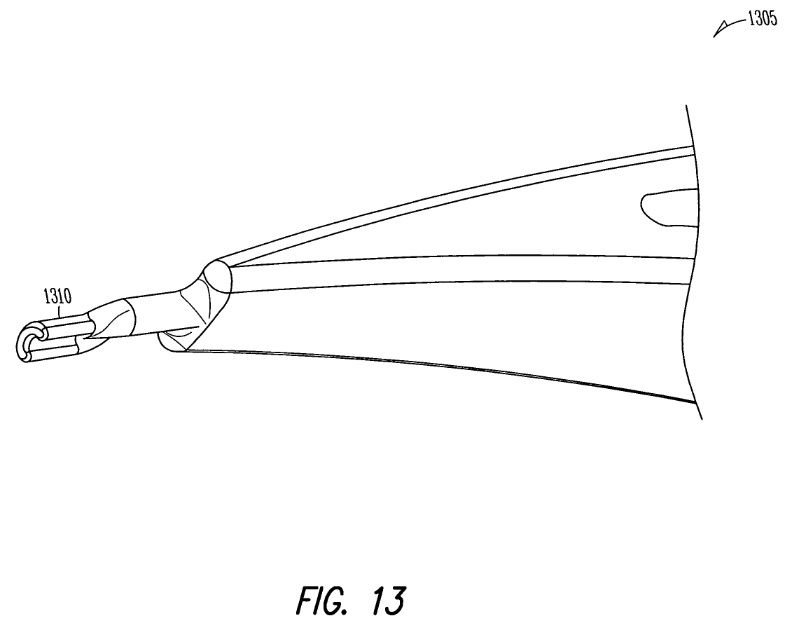

FIG. 13 is a view of an embodiment of the proximal end 1305 of the insertion tool. The proximal end 1305 includes an insertion facilitating portion 1310. The facilitating portion 1310 is configured to facilitate secure insertion of the punctal implant into the punctum. In some embodiments, the insertion facilitating portion 1310 includes a curvature substantially similar to a curvature of at least a portion of the punctal implant. The similar curvature allows manipulation of the punctal implant so that the implant may be securely inserted into the punctum. For example, the curvature may be similar to the drug eluting portion 1245 of the punctal implant 1230 in FIG. 12. The insertion facilitating portion 1310 assists in getting the corner of the punctal implant 1230 into the punctum to lock the punctal implant 1230 in position.

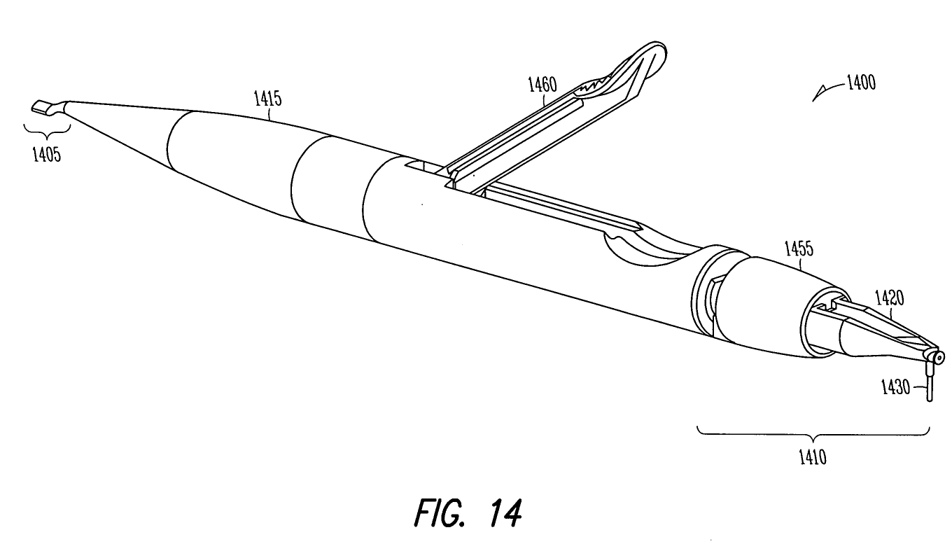

FIG. 14 shows another embodiment of an insertion tool 1400 for use with a punctal implant. The insertion tool 1400 includes a proximal end 1405, a distal end 1410, and a tool body 1415 therebetween. The distal end 1410 includes a forceps 1420. The forceps 1410 are sized to engage a lacrimal implant 1430 on an outer surface of the lacrimal implant 1430. The insertion tool 1400 locks a position of the forceps (e.g., the width of the forceps) when the lacrimal implant 1430 is so engaged.

In some embodiments, the insertion tool 1400 includes a collar 1455. The collar 1455 slidably engages the forceps to cause the forceps to open and close. In certain embodiments, sliding the collar 1455 forward locks the forceps into an engaged position with the lacrimal implant 1430. In some embodiments, the insertion tool 1400 includes a lever 1460 located on the tool body 1415. Manipulating the lever 1460 causes the collar 1455 to slidably engage the forceps 1420. In certain embodiments, lowering or closing the lever 1460 causes the forceps 1420 to close onto the lacrimal implant 1430. In certain embodiments, the lever 1460 is raised or open to close the forceps onto the lacrimal implant 1430, and lowering the lever 1460 then opens the forceps 1420 and releases the lacrimal implant 1430.

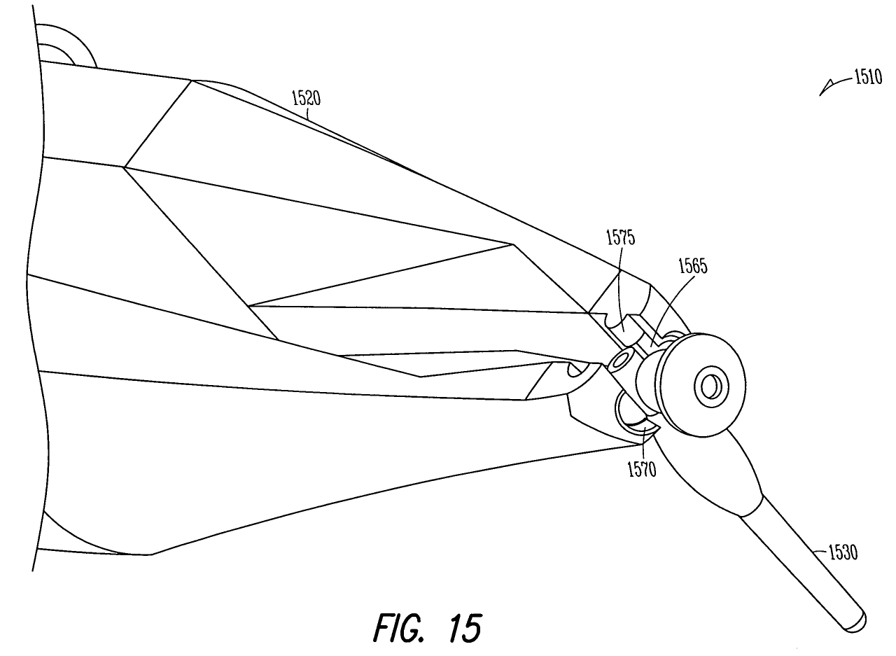

FIG. 15 shows a view of an embodiment of the distal end 1510 of the insertion tool. An end of each arm of the forceps 1520 includes a groove 1565 substantially perpendicular to the forceps arm. The grooves (one for each forceps arm) is sized to receive at least a portion of the lacrimal implant 1530 when the arms of the forceps 1520 are closed. In the embodiment shown, each arm of the forceps includes a first groove 1565 to receive a plug portion of the lacrimal implant 1530 and hold the plug portion perpendicular to the forceps 1520, and a second groove 1570 to receive a drug eluting portion of the lacrimal implant 1530. In the example in the Fig., the lacrimal implant 1530 is an L-shaped self-dilating punctum plug. Different lacrimal implants may use different forceps 1520 to better fit the geometry of different types of implants. In some embodiments, the forceps 1520 are detachable from the tool body and are changeable.

In some embodiments, one or more of the arms of the forceps 1520 includes a stop 1575 or cap to engage an end of the lacrimal implant 1530. In the embodiment shown, one arm of the forceps includes the stop 1575 and the other arm includes a groove to receive the stop. The stop 1575 inhibits movement of the lacrimal implant relative to the forceps 1520. For example, the stop may prevent the lacrimal implant 1530 from sliding in an upward direction when force is applied to the lacrimal implant 1530 by pushing down on the implant. The stop 1575 may also be useful for pushing the lacrimal implant 1530 into the punctum when the forceps are turned over.

Returning to FIG. 14, in some embodiments the proximal end 1405 of the insertion tool 1400 includes an insertion facilitating portion as shown in FIG. 13. If the lacrimal implant is an L-shaped punctum plug, the forceps may be used to insert the longer first portion into the punctum, and the facilitating portion may be used to manipulate the corner of the second transverse portion into the punctum.

The forceps 1420 may be shaped to dilate the punctum for insertion of the lacrimal implant 1430. The forceps 1420 may also be used to extract the lacrimal implant 1430 from the punctum. In some embodiments, the proximal end 1405 may include a second forceps to extract the lacrimal implant. In certain examples, the second set of forceps at the proximal end 1405 are detachable from the insertion tool body 1415.

### Extraction

In some embodiments the implant may include one or more features that may be grasped by an extraction tool to assist in the removal of the implant from the punctum. Embodiments of plugs with one or more features are shown in U.S. Patent Applications 60/970,696, filed on Sep. 7, 2007, titled “EXPANDABLE NASOLACRIMAL DRAINAGE SYSTEM IMPLANTS”, the full disclosures of which are incorporated herein by reference.

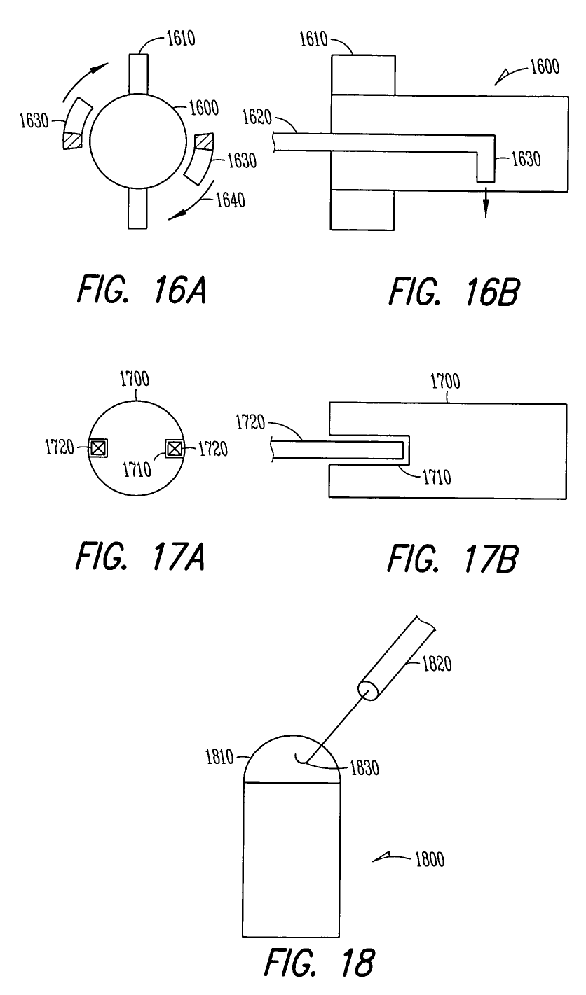

FIGS. 16A and 16B show a top view and side view of an implant 1600 that includes one or more protrusions 1610 or wings and an extraction tool 1620 for removal of the implant 1600 from a punctum of a patient. The extraction tool 1620 may be standard forceps or specialty tool that has angled tips 1630 on a distal end that are curved and configured to grasp the protrusions 1610. The angled tips 1630 are designed such that they can be slid down the side of the implant 1600 (FIG. 16A) and then twisted 1640 under the protrusions 1610 for removal of the implant 1600.

FIGS. 17A and 17B show a top view and side view of an implant 1700 that includes one or more grooves 1710 into which a distal end of an extraction tool 1720 is inserted for removal of the implant. The grooves 1710 may have indentations or other features which couple with the extraction tool 1720. The extraction tool 1720 may be standard forceps or may be a specialty tool designed with mating teeth or other features for engagement with the grooves 1710.

FIG. 18 shows an implant 1800 having a loop or handle 1810 on a top portion that can be grasped an extraction tool 1820 for removal of the implant 1800 from the punctum of a patient. The loop or handle 1810 may be a ribbon or filament positioned across the top of the implant. The extraction tool 1820 may be standard forceps or may be specialty tool having a hook feature 1830 to engage the loop or handle.

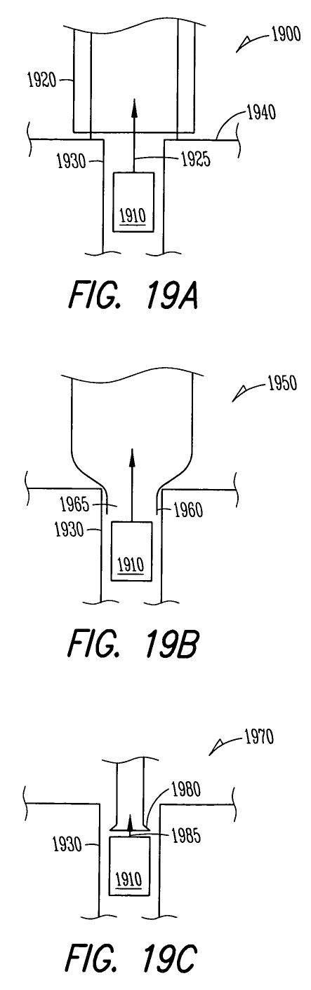

In some embodiments, the extraction tool may be a suction device used for removal of the implant. FIG. 19A shows one embodiment of a suction extraction tool 1900 having a special tip portion 1920 that surrounds the punctum 1930 and seals the tip against the skin 1940. Once in place, a vacuum 1925 is created and the implant 1910 is sucked into the suction device 1900. In one embodiment, the tip is spring-loaded tip or spring-loaded plunger to activate the vacuum, such that the spring must be compressed to turn on the suction feature. In another embodiment, a button or switch associated with the suction extraction tool 1900 may be activated to apply the vacuum. FIG. 19B shows another embodiment of a suction extraction tool 1950 having a tip 1960 configured for insertion into the punctum 1930. Once in place, a vacuum 1965 is created and the implant 1910 is sucked into the suction extraction tool 1950. In some embodiments, the tip 1960 may be similar to the lead-ins or dilators discussed above. In other embodiments, the tip 1960 may be a guidewire having a vacuum lumen. The guidewire may be flexible to negotiate the curves in the canaliculus, allowing it to reach deeper implants. FIG. 19C shows another embodiment of a suction extraction tool 1970 having a suction cup tip 1980 to aid in removal of the implant. The suction cup tip 1980 acts like a plunger on the implant, such that when it is pressed against the implant, a vacuum 1985 is created between the two and then the suction extraction tool 1970 is withdrawn, removing the implant 1910.

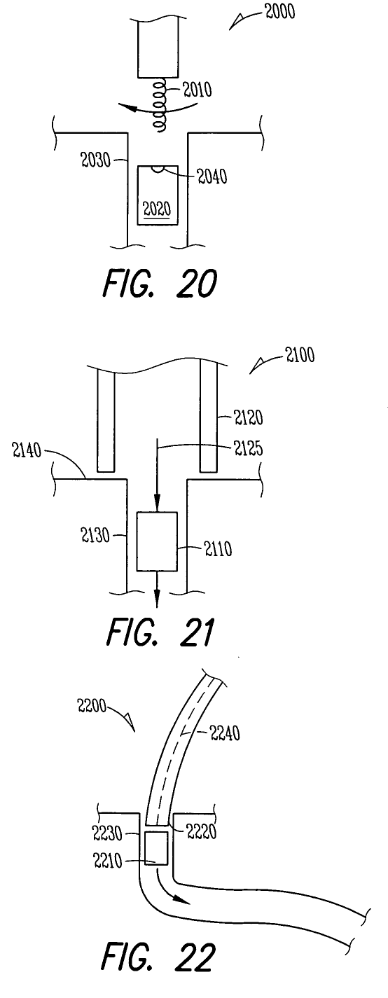

FIG. 20 shows one embodiment of an extraction tool 1500 that includes a helical filament 2010 for implant 2020 for removal of the implant from the punctum of a patient. The helical filament 2010 is a corkscrew like structure and is designed to engage the implant 2020, which should be made of a suitable material, such as silicone, so that the a helical filament 2010 may be easily inserted into the implant 2020 in the punctum 2030. The implant 2020 may also include a hole or depression 2040 to assist in engagement of the helical filament 2010.

In some embodiments, it may be desirable and/or necessary to remove the implant by flushing or pushing the implant through the lacrimal system into the nose and throat. FIG. 21 shows one embodiment of an extraction tool 2100 that is a “flusher” device with a tip portion 2120 positioned proximate the punctum 2130 and engages the skin 2140. The device 2100 uses fluid or air pressure to push the implant 2110 through upper canaliculus 10 or lower canaliculus 12 and into the naso-lacrimal duct 14 (see FIG. 1A). In some embodiments, the tip 2120 may be similar to the lead-in or dilator tips discussed above. FIG. 22 shows one embodiment of an extraction tool 2200 that is a “pusher” device similar to a guidewire having a tip portion 2220 configured to push an implant 2210 through the upper canaliculus 10 or lower canaliculus 12 and into the naso-lacrimal duct 14. The device 2200 may also include an irrigation lumen 2240 that may add a lubricant, e.g. polyethylene glycol (PEG) or polyvinyl alcohol (PVA) demulcent, to aid in removing the implant.

While the exemplary embodiments have been described in some detail, by way of example and for clarity of understanding, those of skill in the art will recognize that a variety of modification, adaptations, and changes may be employed. For example, the above-described examples (or one or more aspects thereof) may be used in combination with each other. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An implant insertion tool for use with a lacrimal implant, the insertion tool comprising:
a proximal end, a distal end, and a tool body therebetween, wherein the distal end includes:
a mechanical coupling to receive a cartridge preloaded with a lacrimal implant; and
a plunger configured to dispense the lacrimal implant from a preloaded cartridge.

2. The insertion tool of claim 1, wherein the cartridge engages an outer surface of the lacrimal implant and contains an inner lumen, wherein the plunger has a diameter greater than or equal to a diameter of a plunger receiving surface of the lacrimal implant and the plunger is configured to slide within the inner lumen and engage and dispense the lacrimal implant from the cartridge.

3. The insertion tool of claim 1, wherein the proximal end includes an insertion facilitating portion.

4. The insertion tool of claim 3, wherein the insertion facilitating portion includes a curvature substantially similar to a curvature of at least a portion of the lacrimal implant.

5. The insertion tool of claim 1, wherein the insertion tool includes a living hinge coupled to the plunger, and wherein manipulating the living hinge causes the plunger to dispense the lacrimal implant.

6. A system for treatment of the eye, comprising:
a lacrimal implant;
a cartridge configured to hold the lacrimal implant;
a lacrimal implant insertion tool for use with the lacrimal implant, the insertion tool comprising:
a proximal end, a distal end, and a tool body therebetween, wherein the distal end includes:
a mechanical coupling to receive the cartridge; and
a plunger configured to dispense the lacrimal implant from the cartridge.

7. The system of claim 6, wherein the plunger has a diameter greater than or equal to a diameter of a plunger receiving surface of the lacrimal implant.

8. The system of claim 7, wherein the lacrimal implant includes a drug eluting portion and a plug portion surrounding at least a portion of the drug eluting portion, and wherein the plunger diameter is greater than or equal to a diameter of the plug portion, and wherein the plunger engages the plug portion to dispense the lacrimal implant.

9. The system of claim 6, wherein the cartridge is rotatable relative to the distal end of the insertion tool.

10. The insertion tool of claim 6, wherein the proximal end of the insertion tool includes an insertion facilitating portion, wherein the insertion facilitating portion includes a curvature substantially similar to a curvature of at least one of the drug eluting portion and the plug portion of the lacrimal implant.

11. An implant insertion tool for use with a lacrimal implant, the insertion tool comprising:
a proximal end, a distal end, and a tool body therebetween;
a forceps located at the distal end, wherein the forceps are sized to engage the lacrimal implant on an outer surface of the implant, and wherein the insertion tool is configured to lock a position of the forceps when the lacrimal implant is so engaged.

12. The insertion tool of claim 11, including a collar to slidably engage the forceps to cause the forceps to open and close.

13. The insertion tool of claim 12, including a lever located on the tool body, wherein manipulating the lever causes the collar to slidably engage the forceps.

14. The insertion tool of claim 11, wherein an end of each arm of the forceps includes a groove substantially perpendicular to the forceps arm, wherein the grooves are sized to receive at least a portion of the lacrimal implant when the forceps are closed.

15. The insertion tool of claim 14, wherein at least one of the forceps arms includes a stop to engage an end of the lacrimal implant and inhibit movement of the lacrimal implant relative to the forceps.

16. The insertion tool of claim 11, wherein the proximal end includes an insertion facilitating portion.

17. The insertion tool of claim 11, wherein the proximal end of the insertion tool includes a second forceps configured to extract the lacrimal implant from the punctum.

18. The insertion tool of claim 11, wherein the forceps are detachable from the tool body.

19. A method of inserting an implant using an insertion tool, the method comprising:
preloading a lacrimal implant into a cartridge; and
dispensing the lacrimal implant from the cartridge to insert the lacrimal implant into a punctum.

20. The method of claim 19, wherein preloading a lacrimal implant into a cartridge includes engaging an outer surface of the lacrimal implant to releasably support the lacrimal implant.

21. The method of claim 19, wherein dispensing the lacrimal implant includes dispensing the lacrimal implant from the cartridge using a plunger.

22. The method of claim 21, including manipulating a living hinge on the insertion tool to engage the lacrimal implant with the plunger.

23. A method of inserting an implant using an insertion tool, the method comprising:
engaging an outer surface of the lacrimal implant with a forceps;
locking a forceps position when the outer surface of the lacrimal implant is engaged; and
advancing the lacrimal implant into a punctum.

24. The method of claim 23, including slidably engaging arms of the forceps with a collar to open and close the forceps.

25. The method of claim 24, wherein slidably engaging arms of the forceps with a collar includes manipulating a lever to cause the collar to slidably engage the arms of the forceps.

26. The method of claim 24, including receiving the lacrimal implant into a groove on a forcep arm when the forceps are closed, wherein the groove is substantially perpendicular to the forcep arm and is sized to receive the lacrimal implant.

27. The method of claim 23, wherein advancing the lacrimal implant into the punctum includes engaging an end of the lacrimal implant with a stop on a forcep arm to inhibit movement of the lacrimal implant relative to the forcep arm.

28. The method of claim 23, including changing the forceps of the insertion tool to fit a geometry of the lacrimal implant.

29. An implant insertion tool for insertion of a lacrimal implant into a punctum of a patient, the insertion tool comprising:
a tool body having a distal portion configured to hold the implant on an outer implant surface, the distal portion having an inner lumen with an internal depth stop; and
a plunger slidable within the inner lumen to engage and dispense the implant, the plunger having a stop configured to engage with the internal depth stop, wherein the engagement of the stop and the internal depth stop limits an insertion depth of the implant into the punctum.

30. An implant insertion tool for use with a lacrimal implant, the insertion tool comprising:
a tool body having a proximal handle, a distal end, and an axis therebetween, the tool body including:
an implant receptacle releasably supporting, on at least one outer implant surface, the lacrimal implant relative to the handle, such that the lacrimal implant is advanceable distally into a canalicular lumen by manipulation of the handle; and
a tissue-engagement stop surface, the stop surface being distally oriented and configured to engage an anteriorly oriented tissue surface to inhibit distal insertion of the lacrimal implant beyond a target insertion depth.

31. A lacrimal implant insertion system for treatment of one or more tissues near a punctum of a patient, the punctum disposed between a canalicular lumen and an anteriorly oriented tissue surface of the patient, the lacrimal implant system comprising:
a self-dilating lacrimal implant; and
an insertion tool having a proximal handle, a distal implant receptacle, and an axis therebetween, the implant receptacle releasably supporting the lacrimal implant such that the lacrimal implant is advanceable distally into the canalicular lumen by manipulation of the handle, the insertion tool including a tissue-engagement stop surface, the stop surface being distally oriented and configured to engage the anteriorly oriented tissue surface so as to inhibit distal insertion of the lacrimal implant beyond a target insertion depth.

32. A method of inserting an implant into a punctum of a patient using an insertion tool, the method comprising:
advancing the implant distally into the punctum;
engaging a tissue stop of the insertion tool against a tissue surface of the punctum so as to inhibit distal movement of the insertion tool; and
detaching the implant from the insertion tool while the tissue stop engages the tissue surface and while the implant is aligned axially relative to the tissue stop so that the implant is implanted at a target depth within the canalicular lumen.

33. A method of inserting an implant into a punctum of a patient using an insertion tool, the method comprising:
placing a tissue stop of the insertion tool proximate the punctum;
moving a plunger within the insertion tool forward, thereby inserting the implant into the punctum; and
stopping the plunger movement when a stop on the plunger engages an internal depth stop of the insertion tool, wherein the engagement of the stop and internal depth stop limits the depth of insertion of the implant into the punctum.

34. An extraction tool for extraction of an implant from a punctum of a patient, the extraction tool comprising:
an extraction tool body having a distal portion, wherein the distal portion includes an extraction feature to engage a complimentary extraction feature of the implant.

35. An extraction tool for extraction of an implant from a punctum of a patient, the extraction tool comprising:
an extraction tool body having a distal portion; and
a suction device configured to provide a suction force to the extraction tool body, wherein the distal portion of the extraction tool includes an inner lumen extending to a tip of the distal portion, and wherein the tip is configured to engage the punctum and apply the suction force to extract the implant.
